(19) United States
(12) Patent Application Publication
(10) Pub. No.: US 2020/0138456 A1
(43) Pub. Date: May 7, 2020

(54) ALIGNMENT GUIDE FOR REAMER SHAFT

(71) Applicant: Avalign Technologies, Inc., Bannockburn, IL (US)

(72) Inventor: Warren Scott Gareiss, Warsaw, IN (US)

(21) Appl. No.: 16/183,255

(22) Filed: Nov. 7, 2018

(51) Int. Cl. A61B 17/17

(52) U.S. Cl. CPC A61B 17/17

(57) ABSTRACT

An alignment guide is provided for aligning a cannulated intramedullary reamer shaft with a guidewire. The alignment guide includes a tray, a guidewire alignment flange extending from a rear portion of the tray, and a reamer shaft alignment flange extending from a front portion of the tray. The guidewire alignment flange includes a sidewall curved to form a first recess within the sidewall. The reamer shaft alignment flange includes at least one tab having a curved portion to form a second recess within the at least one tab. The first and second recesses are aligned along a longitudinal axis to position the reamer shaft and guidewire along the longitudinal axis.

### BACKGROUND

In some instances, an anatomic region of the human anatomy may need to be repaired and/or replaced due to injury, stress, degenerative health issues, or various other problems. To perform such a repair and/or replacement on the affected anatomical region, it may be necessary to cut or remove portions of bone and associated tissue matter with a reaming device, or reamer, as shown in FIG. 1. A reamer (50) typically includes a continuous reamer shaft (52) with a reamer tip (54) at the end of the reamer shaft (52) used to cut or bore into the inner canal of a long bone (2) (e.g., femur, tibia, etc.) to clean and create space for an implant to be received and/or to help mend a fractured bone. Before the reaming process, a guidewire (40) is typically inserted into the space to serve as a guide for receiving the reamer shaft (52).

Typically, the reamer shaft (52) is positioned over the guidewire (40) by inserting the guidewire (40) into an opening through the longitudinal axis of the reamer shaft (52). To insert the guidewire (40) into the reamer shaft (52), the tip of the guidewire (44) must be aligned with the tip (54) of the reamer shaft (52). As shown in FIG. 1, the guidewire (40) and/or the reamer shaft (52) may be flexible such that the portion of the guidewire (40) extending from the bone (2) and/or the reamer shaft (52) may bend back and forth, making it difficult to align the tip (44) of the guidewire (40) with the tip (54) of the reamer shaft (52). Accordingly, it may be desirable to provide an alignment guide to more easily and efficiently align a reamer shaft with a guidewire.

While a variety of alignment guides have been made and used, it is believed that no one prior to the inventors has made or used an invention as described herein.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a schematic of an exemplary reamer shaft being aligned with an exemplary guidewire without an alignment guide.

FIG. 2 depicts a schematic of the reamer shaft and guidewire of FIG. 1 aligned via an exemplary alignment guide.

FIG. 3 depicts a perspective view of the alignment guide of FIG. 2.

FIG. 4 depicts a cross-sectional view of the alignment guide of FIG. 3 taken along line 4-4 of FIG. 3.

FIG. 5 depicts a front view of a reamer shaft alignment tab of the alignment guide of FIG. 3.

FIG. 6 depicts a front view of the alignment guide of FIG. 3.

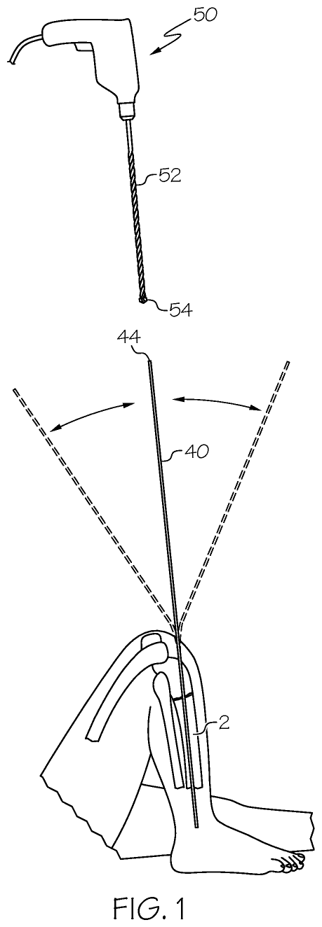

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It will be appreciated that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Alignment Guide

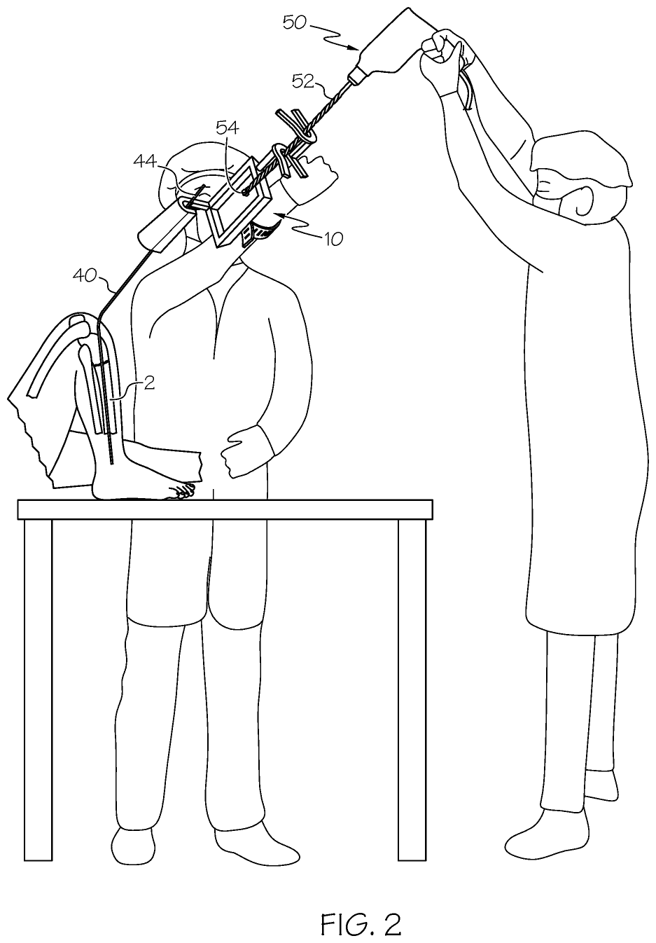

In some instances, it may be desirable to provide an alignment guide to aide in the placement of an intramedullary reamer over a guidewire. Referring to FIG. 2, an alignment guide (10) is shown being used to maintain the position of the tip (44) of the guidewire (40) relative to the position of the tip (54) of the reamer shaft (52) to insert the guidewire (40) within the reamer shaft (52). Accordingly, the alignment guide (10) may prevent the flexible guidewire (40) from bending back and forth while the guidewire (40) is being positioned relative to the reamer shaft (52), allowing the reamer shaft (52) thereby be placed more easily and more efficiently over the guidewire (40), as will be discussed in more detail below.

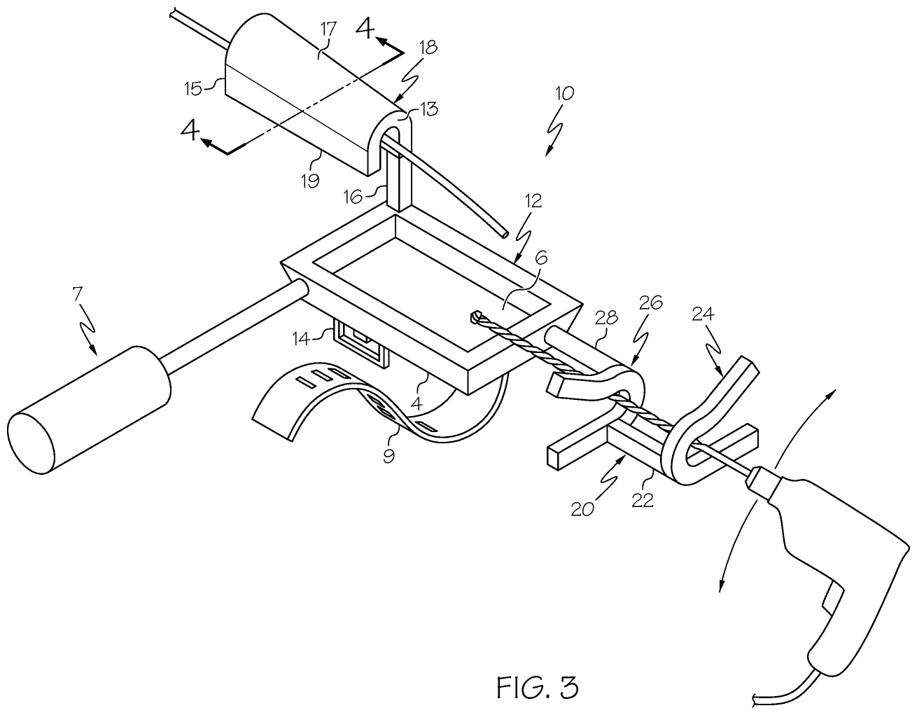

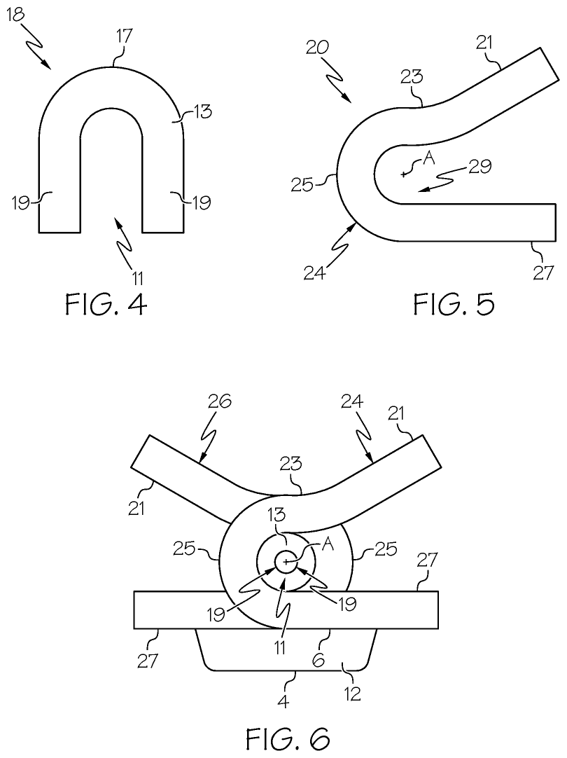

Referring to FIG. 3, an alignment guide (10) is shown comprising a tray (12) positioned between a guidewire alignment flange (18) and a reamer shaft alignment flange (20) aligned along a longitudinal axis (A). The alignment guide (10) may be made from plastic or other suitable material such that the alignment guide (10) is sufficiently rigid and light weight. The guidewire alignment flange (18), as shown in FIG. 4, comprises a sidewall (17) having a front end (13) and a rear end (15). The sidewall (17) is curved into an arcuate profile to form a recess (11) in the interior portion of the sidewall (17) between a pair of bottom surfaces (19). In the illustrated embodiment, the curved sidewall (17) is tapered such that the thickness of the front end (13) is less than the thickness of the rear end (15). Accordingly, a top surface of the sidewall (17) may be level relative to the tray (12) such that the rear end (15) of the sidewall (17) extends downward with respect to the front end (13), as shown in FIG. 4. A front portion of the guidewire alignment flange (18) in the illustrated embodiment is coupled to a rear portion of the tray (12) by a guidewire alignment flange shaft (16) extending upwardly from the tray (12), as shown in FIG. 3.

The tip (44) of the guidewire (40) may thereby be inserted through the recess (11) of the guidewire alignment flange (18) from the rear end (15) to the front end (13) to position the tip (44) of the guidewire (40) above a top surface (6) of the tray (12). The taper of the sidewall (17) may guide the guidewire (40) into a level position above the tray (12) such that the guidewire (40) may extend along the longitudinal axis (A), substantially parallel with the tray (12). The radius of the recess (11) of the sidewall (17) may correspond to the outer diameter of the guidewire (40) such that the sidewall (17) maintains the horizontal position of the guidewire (40) relative to the tray (12), but may still allow the guidewire (40) to move vertically within the sidewall (17) relative to the tray (12). Still other suitable configurations for the guidewire alignment flange (18) will be apparent to one with ordinary skill in the art in view of the teachings herein.

FIG. 3 further shows the reamer shaft alignment flange (20) comprising a front tab (24) and a rear tab (26) separated by a shaft (22). Referring to FIG. 5, each tab (24, 26) comprises a tapered portion (21) extending upwardly and outwardly from a flat portion (23). The flat portion (23) may be positioned substantially parallel with the tray (12). A curved portion (25) may then extend from the other end of the flat portion (23) to form a circular profile about the longitudinal axis (A). A bottom portion (27) then extends from the other end of the curved portion (25) underneath the flat portion (23) and the tapered portion (21). A recess (29) is thereby formed between the flat portion (23) and the bottom portion (27). In the illustrated embodiment, the bottom portion (27) is also positioned substantially parallel relative to the tray (12). Still other suitable configurations for the tabs (24, 26) will be apparent to one of ordinary skill in the art in view of the teachings herein.

Referring back to FIG. 3, the front tab (24) is positioned to extend in an opposing direction relative to the rear tab (26). The rear tab (26) may then be coupled with a front portion of the tray (12) by shaft (28) to align the bottom portion (27) of each tab (24, 26) with the top surface (6) of the tray (12), as shown in FIG. 6. The tip (54) of the reamer shaft (52) may thereby be inserted through the recess (29) of each tab (24, 26) to position the tip (54) of the reamer shaft (52) above the top surface (6) of the tray (12). The tapered portion (21) of each tab (24, 26) may guide the reamer shaft (52) into the recess (29) of each tab (24, 26) such that the reamer shaft (52) may extend along the longitudinal axis (A), substantially parallel with the tray (12). The radius of the recess (29) of each tab (24, 26) may correspond to the outer diameter of the reamer shaft (52) such that the tabs (24, 26) maintain the vertical position of the reamer shaft (52) relative to the tray (12), but may still allow the reamer shaft (52) to move horizontally within the flat portion (23) of each tab (24, 26) relative to the tray (12). Still other suitable configurations for the reamer shaft alignment flange (20) will be apparent to one with ordinary skill in the art in view of the teachings herein.

Accordingly, as shown in FIG. 6, the guidewire alignment flange (18) and the reamer shaft alignment flange (20) are aligned along the longitudinal axis (A). The front tab (24) and the rear tab (26) of the reamer shaft alignment flange (20) are oriented relative to each other such that the curved portions (25) of the tabs (24, 26) form an opening about the longitudinal axis (A) having a diameter that may be slightly larger than an outer diameter of the reamer shaft (52) to maintain the position of the reamer shaft (52) along the longitudinal axis (A) when the reamer shaft (52) is inserted within the reamer shaft alignment flange (20). The front end (13) of the guidewire alignment flange (18) may then be aligned within the opening formed by the tabs (24, 26) of the reamer shaft alignment flange (20). Because the outer diameter of the guidewire (40) is smaller than the outer diameter of the reamer shaft (52) to allow the guidewire (40) to be inserted within the reamer shaft (52), the recess (11) of the guidewire alignment flange (18) may be smaller than the opening formed by the tabs (24, 26) of the reamer shaft alignment flange (20). The guidewire alignment flange (18) may thereby maintain the position of the guidewire (40) along the longitudinal axis (A) when the guidewire (40) is inserted within the guidewire alignment flange (18).

The tray (12) of the illustrated embodiment comprises a strap (9), with a first end of the strap (9) attached to one side of a bottom surface (4) of the tray (12). The other side of the bottom surface (4) of the tray (12) comprises a hook (14) such that the free end of the strap (9) may be inserted through the hook (14) and wrapped around to the exterior surface of the strap (9). The free end of the strap (9) may then be selectively secured to the exterior surface of the strap (9) by a fastener, such as a hook and loop fastener, a snap, a button, or other suitable fastener. The strap (9) may thereby allow the alignment guide (10) to be selectively coupled with a surgeon or other medical technician. In some versions, the alignment guide (10) may comprise a handle (7) extending outwardly from the tray (12) that may be grasped by a medical technician to position the alignment guide (10) either in addition to the strap (9) or instead of the strap (9). Still other suitable configurations for the alignment guide (10) will be apparent to one with ordinary skill in the art in view of the teachings herein.

II. Method of Operating an Alignment Guide

In use, the alignment guide (10) may aide in the placement of an intramedullary reamer shaft (52) over a guidewire (40) as shown in FIG. 2. For instance, the guidewire (40) may be inserted within a bone (2) for reaming. The alignment guide (10) may be selectively coupled with a surgeon or other medical technician by placing the bottom surface (4) of the tray (12) against a wrist and/or forearm of the user. The strap (9) may then be wrapped about the wrist and/or forearm of the user, through the hook (14), and secured to itself to hold the alignment guide (10) in place. The tip (44) of the guidewire (40) may then be inserted through the recess (11) of the guidewire alignment flange (18) from the rear end (15) to the front end (13) to position the tip (44) of the guidewire (40) above a top surface (6) of the tray (12). The guidewire alignment flange (18) may guide the guidewire (40) into a level position above the tray (12) such that the guidewire (40) may extend along the longitudinal axis (A). The user may orient the guidewire (40) axially behind and to the side of the user, where a surgical technician may be standing.

The surgical technician may then position the tip (54) of the reamer shaft (52) over the top surface (6) of the tray (12) and rotate the reamer shaft (52) into the tabs (24, 26) of the reamer shaft alignment flange (20). For instance, each tab (24, 26) of the reamer shaft alignment flange (20) may guide the reamer shaft (52) into the recess (29) of each tab (24, 26) such that the reamer shaft (52) may extend along the longitudinal axis (A), substantially parallel with the tray (12). With both the guidewire (40) and the reamer shaft (52) aligned along the longitudinal axis (A), the reamer shaft (52) may be translated toward the guidewire (40) to more easily position the reamer shaft (52) over the guidewire (40). For instance, the tip (44) of the guidewire (40) may be maintained in the alignment guide (10) during insertion within the reamer shaft (52) to prevent the guidewire (40) from bending back and forth. Further, with the alignment guide (10) positioned on one hand and/or arm of the user, the other hand of the user may be used to support the reamer shaft (52) and/or the reamer (50) as needed instead of trying to align the guidewire (40) with the reamer shaft (52). This may also reduce the time needed to perform the reaming procedure, especially in procedures where sequential reaming is used. Still other suitable configurations for using the alignment guide (10) to align the guidewire (40) with the reamer shaft (52) will be apparent to one with ordinary skill in the art in view of the teachings herein.

III. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An alignment guide comprising:
(a) a guidewire alignment flange comprising a sidewall curved to form a first recess within the sidewall; and
(b) a reamer shaft alignment flange comprising a front tab and a rear tab, wherein the front tab comprises a curved portion to form a second recess within the front tab, wherein the rear tab comprises a curved portion to form a third recess within the rear tab, wherein the front tab and the rear tab are oriented in opposite directions;

wherein the first, second, and third recesses are aligned along a longitudinal axis.

### Example 2

The alignment guide of example 1, wherein the sidewall of the guidewire alignment flange is tapered.

### Example 3

The alignment guide of example 2, wherein a rear end of the sidewall is thicker than a front end of the sidewall.

### Example 4

The alignment guide of any of the examples 1 to 3, wherein the guidewire alignment flange is configured to receive a guidewire through the first recess to position the guidewire along the longitudinal axis.

### Example 5

The alignment guide of example 4, wherein an inner diameter of the first recess corresponds to an outer diameter of the guidewire such that the horizontal position of the guidewire is maintained relative to the guidewire alignment flange when the guidewire is inserted within the first recess.

### Example 6

The alignment guide of any of the examples 1 to 5, wherein each of the front tab and the rear tab of the reamer shaft alignment flange comprises a flat portion and a bottom portion extending from the curved portion such that the flat portion is substantially parallel to the bottom portion.

### Example 7

The alignment guide of example 6, wherein each of the front tab and the rear tab further comprise a tapered portion extending upwardly and outwardly from the flat portion.

### Example 8

The alignment guide of any of the examples 1 to 8, wherein the reamer shaft alignment flange is configured to receive a reamer shaft through the second recess and the third recess to position the reamer shaft along the longitudinal axis.

### Example 9

The alignment guide of example 8, wherein an inner diameter of the second recess and the third recess corresponds to an outer diameter of the reamer shaft such that the vertical position of the reamer shaft is maintained relative to the reamer shaft alignment flange when the reamer shaft is inserted within the second recess and the third recess.

### Example 10

The alignment guide of any of the examples 1 to 9 further comprising a tray positioned between the guidewire alignment flange and the reamer shaft alignment flange.

### Example 11

The alignment guide of example 10, wherein a top surface of the tray is aligned with a bottom portion of the reamer shaft alignment flange.

### Example 12

The alignment guide of examples 10 or 11, wherein the tray comprises a strap to selectively couple the tray with an arm of a user.

### Example 13

The alignment guide of examples 10 or 11, wherein the tray comprises a handle for grasping by a user.

### Example 14

An alignment guide comprising:
(a) a tray;
(b) a guidewire alignment flange extending from a rear portion of the tray, wherein the guidewire alignment flange comprises a sidewall curved to form a first recess within the sidewall;
(c) a reamer shaft alignment flange extending from a front portion of the tray, wherein the reamer shaft alignment flange comprises at least one tab, wherein the at least one tab comprises a curved portion to form a second recess within the at least one tab, wherein the first and second recesses are aligned along a longitudinal axis.

### Example 15

A method of operating an alignment guide comprising the steps of:
(a) inserting a guidewire into a guidewire alignment flange of the alignment guide such that the guidewire alignment flange guides the position of the guidewire along a longitudinal axis; and
(b) inserting a reamer shaft into a reamer shaft alignment flange of the alignment guide such that the reamer shaft alignment flange guides the position of the reamer shaft along the longitudinal axis; and
(c) translating the reamer shaft toward the guidewire to insert a tip of the reamer shaft over a tip of the guidewire.

### Example 16

The method of example 15, wherein the guidewire is inserted into a recess defined by a curved sidewall of the guidewire alignment flange.

### Example 17

The method of examples 15 or 16, wherein the reamer shaft is inserted into a recess defined by a curved portion of a tab of the reamer shaft alignment flange.

### Example 18

The method of any of the examples 15 to 17 further comprising aligning the tip of the guidewire with the tip of the reamer shaft above a top surface of a tray before the reamer shaft is translated toward the guidewire.

### Example 19

The method of example 18 further comprising positioning a bottom surface of the tray against an arm of a user and wrapping a strap of the tray around the arm to selectively couple the tray with the user.

### Example 20

The method of example 18 further comprising positioning a bottom surface of the tray against an arm of a user and grasping a handle to hold the tray against the user.

IV. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An alignment guide comprising:
(a) a guidewire alignment flange comprising a sidewall curved to form a first recess within the sidewall; and
(b) a reamer shaft alignment flange comprising a front tab and a rear tab, wherein the front tab comprises a curved portion to form a second recess within the front tab, wherein the rear tab comprises a curved portion to form a third recess within the rear tab, wherein the front tab and the rear tab are oriented in opposite directions;
wherein the first, second, and third recesses are aligned along a longitudinal axis.

2. The alignment guide of claim 1, wherein the sidewall of the guidewire alignment flange is tapered.

3. The alignment guide of claim 2, wherein a rear end of the sidewall is thicker than a front end of the sidewall.

4. The alignment guide of claim 1, wherein the guidewire alignment flange is configured to receive a guidewire through the first recess to position the guidewire along the longitudinal axis.

5. The alignment guide of claim 4, wherein an inner diameter of the first recess corresponds to an outer diameter of the guidewire such that the horizontal position of the guidewire is maintained relative to the guidewire alignment flange when the guidewire is inserted within the first recess.

6. The alignment guide of claim 1, wherein each of the front tab and the rear tab of the reamer shaft alignment flange comprises a flat portion and a bottom portion extending from the curved portion such that the flat portion is substantially parallel to the bottom portion.

7. The alignment guide of claim 6, wherein each of the front tab and the rear tab further comprise a tapered portion extending upwardly and outwardly from the flat portion.

8. The alignment guide of claim 1, wherein the reamer shaft alignment flange is configured to receive a reamer shaft through the second recess and the third recess to position the reamer shaft along the longitudinal axis.

9. The alignment guide of claim 8, wherein an inner diameter of the second recess and the third recess corresponds to an outer diameter of the reamer shaft such that the vertical position of the reamer shaft is maintained relative to the reamer shaft alignment flange when the reamer shaft is inserted within the second recess and the third recess.

10. The alignment guide of claim 1 further comprising a tray positioned between the guidewire alignment flange and the reamer shaft alignment flange.

11. The alignment guide of claim 10, wherein a top surface of the tray is aligned with a bottom portion of the reamer shaft alignment flange.

12. The alignment guide of claim 10, wherein the tray comprises a strap to selectively couple the tray with an arm of a user.

13. The alignment guide of claim 10, wherein the tray comprises a handle for grasping by a user.

14. An alignment guide comprising:
(a) a tray;
(b) a guidewire alignment flange extending from a rear portion of the tray, wherein the guidewire alignment flange comprises a sidewall curved to form a first recess within the sidewall;
(c) a reamer shaft alignment flange extending from a front portion of the tray, wherein the reamer shaft alignment flange comprises at least one tab, wherein the at least one tab comprises a curved portion to form a second recess within the at least one tab, wherein the first and second recesses are aligned along a longitudinal axis.

15. A method of operating an alignment guide comprising the steps of:
(a) inserting a guidewire into a guidewire alignment flange of the alignment guide such that the guidewire alignment flange guides the position of the guidewire along a longitudinal axis;
(b) inserting a reamer shaft into a reamer shaft alignment flange of the alignment guide such that the reamer shaft alignment flange guides the position of the reamer shaft along the longitudinal axis; and
(c) translating the reamer shaft toward the guidewire to insert a tip of the reamer shaft over a tip of the guidewire.

16. The method of claim 15, wherein the guidewire is inserted into a recess defined by a curved sidewall of the guidewire alignment flange.

17. The method of claim 15, wherein the reamer shaft is inserted into a recess defined by a curved portion of a tab of the reamer shaft alignment flange.

18. The method of claim 15 further comprising aligning the tip of the guidewire with the tip of the reamer shaft above a top surface of a tray before the reamer shaft is translated toward the guidewire.

19. The method of claim 18 further comprising positioning a bottom surface of the tray against an arm of a user and wrapping a strap of the tray around the arm to selectively couple the tray with the user.

20. The method of claim 18 further comprising positioning a bottom surface of the tray against an arm of a user and grasping a handle to hold the tray against the user.
